(19) United States
(12) Patent Application Publication
(10) Pub. No.: US 2003/0192067 A1
(43) Pub. Date: Oct. 9, 2003

(54) Delivery system

(75) Inventors: Robert E. Sinden, London (GB); Julian M. Crampton, Liverpool (GB)

Correspondence Address: NIXON & VANDERHYE, PC, 1100 N GLEBE ROAD, 8TH FLOOR, ARLINGTON, VA, 22201-4714, US

(73) Assignee: Imperial College of Science Technology and Medicine

(21) Appl. No.: 10/375,050

(22) Filed: Feb. 28, 2003

Related U.S. Application Data
(63) Continuation of application No. 09/882,302, filed on Jun. 18, 2001, now abandoned.
Application No. 09/882,302 is a continuation of application No. 09/373,225, filed on Aug. 12, 1999, now abandoned.
Application No. 09/373,225 is a continuation-in-part of application No. 08/836,345, filed on Jul. 11, 1997, now abandoned.

(30) Foreign Application Priority Data
Nov. 11, 1994 (GB) 9422827.7
Nov. 10, 1995 PCT/GB95/02639

(51) Int. Cl. A01K 67/033; A61K 39/00

(52) U.S. Cl. 800/8; 424/184.1

(57) ABSTRACT

The invention relates to a delivery system for a recipient, the delivery system comprising a transgenic organism containing a vaccine for the recipient, wherein the vaccine is expressed by a gene contained within the transgenic organism, and the vaccine is capable of being transmitted by and from the organism to the recipient, and wherein the vaccine is prepared outside of the recipient, and wherein the recipient is an animal.

The present invention relates to a delivery system.

In particular the present invention relates to a vaccine delivery system for delivering a vaccine to a recipient—such as a human.

More in particular, the present invention relates to a delivery system for delivering parasite antigens to recipients by organisms, such as phytophagous or haematophagous organisms, such as organisms that usually harbour parasites.

Even more in particular, the present invention relates to a delivery system for delivering malarial vaccines to recipients by mosquitos.

General teachings on the transgenic technology of insect vectors of animal and human disease may be found in Crampton et al (1989).

Amongst the problems encountered in the effective immunisation of human populations in tropical regions are the logistics of vaccine delivery, in particular repeated vaccine delivery, and the effective targeting of appropriate recipients. An example of the former is the anti-malarial transmission-blocking vaccine (TBV25H) which is about to undergo field trials. Here it has been suggested that effective immunity will need to be sustained for up to about 12 years if effective control of the parasite is ever to be achieved in endemic areas. However, immunised patients will not be protected from infection, and the resulting parasitaemias will not boost immunity (Kaslow 1992; Sinden, 1994). This raises the problem of how to sustain immunity effectively.

In this regard, there have been some attempts to overcome this problem—such as designing sustained release immunogens (Kaslow, 1992)—but whilst these approaches may appear to be promising they fall short of the required theoretical goal.

Furthermore, infections with alternative reservoir hosts e.g. T. cruzi, Leishmania and the veterinary parasites Theleria and Babesia present particular problems that need to be overcome if effective protection of endemic populations is to be achieved. For such cases it would be desirable, but at present impossible, to immunise both the human/bovine/equine recipients and reservoir populations in parallel. Moreover, the reservoir is often not readily immunised or identified.

Therefore, in summation, many vaccination programmes aimed at the effective immunisation of human and agricultural animal populations encounter major problems in the logistics of sustained vaccine delivery and the efficient targeting of appropriate recipients.

Targeting is a particular problem in the case of parasitic diseases with alternative reservoir hosts.

The present invention seeks to overcome the problems associated with the known vaccination programmes.

According to a first aspect of the present invention there is provided a delivery system for a recipient, the delivery system comprising an organism containing a vaccine for the recipient, wherein the vaccine is capable of being transmitted by and from the organism to the recipient, and wherein the vaccine is prepared outside of the recipient.

According to a second aspect of the present invention there is provided a transgenic organism comprising a gene capable of expressing a vaccine wherein the transgenic organism itself is suitable for use as a means of delivering the vaccine to a recipient for the vaccination thereof, and wherein the vaccine is prepared outside of the recipient.

According to a third aspect of the present invention there is provided a method of vaccinating a recipient comprising exposing the recipient to a transgenic organism according to the present invention and allowing the vaccine to be transmitted by and from the transgenic organism to the recipient, wherein the vaccine is prepared outside of the recipient.

According to a fourth aspect of the present invention there is provided a vaccine derived from a transgenic organism according to the present invention.

According to a fifth aspect of the present invention there is provided the use of a transgenic organism according to the present invention for the manufacture of a vaccine for the vaccination of a recipient, wherein the vaccine is delivered to the recipient by the transgenic organism, and wherein the vaccine is prepared outside of the recipient.

According to a sixth aspect of the present invention there is provided a transgenic insect (such as a transgenic haematophagous or phytophagous insect) having salivary glands comprising a gene capable of expressing a desired antigen.

According to a seventh aspect of the present invention there is provided a recipient comprising the saliva of a transgenic organism according to the present invention wherein the saliva contains an antigen produced by expression of the gene coding for the same.

According to an eighth aspect of the present invention there is provided the use of a transgenic organism according to the present invention for the manufacture of, and the delivery of, a vaccine for the vaccination of a recipient, wherein the vaccine is prepared outside of the recipient and is delivered to the recipient by the transgenic organism.

The term “recipient” includes any suitable subject to which a suitable vaccine is to be delivered by use of the delivery system of the present invention.

For example, the recipient could be a human or a livestock animal—such as a horse or a cow. It may even be a plant. The term “recipient” also includes a collection (i.e. a reservoir) of subjects.

The term “organism” includes any organism that can deliver a suitable vaccine to a desired recipient. Examples of organisms include phytophagous or haematophagous organisms harbouring a parasites—such as mosquitos, sand flies, stable flies, ticks, Tsetse flies etc. Other examples include aphids and other plant feeding insects.

Preferably, the organism is an organism which in its native state would normally feed on the desired recipient.

Thus, a preferred embodiment is a mosquito containing a malarial vaccine.

The organism may be an organism which in its native state would normally feed on a desired recipient and in doing so transfer infectious agents to the recipient thereby causing infection.

The synonymous terms “vaccine” and “antigen” are used in their normal sense—i.e. an entity (such as a protein) having the ability, post administration to an organism, to stimulate the production of an appropriate response and thus achieve immunity (partial or complete) to the relevant infection.

Examples of vaccines include those that yield immunity against malaria, leishmaniasis, sleeping sickness, or bilharzia etc.

The term “transgenic organism” means an organism containing an additional gene or additional genes which is (are) typically non-natural to the organism. Preferably, the additional gene(s) is (are) non-natural genes to the organism—such as a gene coding for a malarial vaccine. If the gene that is capable of expressing the vaccine is stably incorporated into the genome of the transgenic organism then progeny of the organism will also have the ability to express the vaccine. This is particularly advantageous.

Preferably the organism is a transgenic organism and the vaccine is expressed by a gene contained within the transgenic organism.

The gene may be present in the transgenic organism in, for example, a plasmid or other form. Preferably, however, the gene is stably incorporated in the genome of the transgenic organism.

Preferably the recipient is a livestock animal, a human or a plant.

Preferably the organism has a mouth and the vaccine is transmitted via the mouth of the organism.

Preferably the organism is an phytophagous or haematophagous organism.

Preferably the organism is an insect.

Preferably the organism is an haematophagous insect.

Preferably the organism is a mosquito.

Preferably the mosquito is of the species Culer or Aedes or Anopheles.

Preferably the antigen is a malarial vaccine.

Preferably the organism is an organism that usually harbours a parasite.

Preferably the gene codes for an antigen that affects (e.g. stops or at least hinders) the life cycle of a parasite causing the disease to be treated. The gene may code for a transmission-blocking immunogen, such as TBV25H or Pfs28 (Duffy, P. E. and Kaslow, D. C (1997) Infect.Immun. vol. 65 pp1109-13; Duffy, P. E., Pimenta, P. and Kaslow, D. C. (1993) J.Exp.Med. vol. 177 pp505-10; Gozar, M. M. G., Price, V. L. and Kaslow, D. C. (1998) lnfect.Immun. vol.66 pp59-64; Kaslow, D. C. and Shiloach, J. (1994) Biotechnology vol.12 pp494-9).

A preferable combination of the present invention includes a livestock animal, a human or a plant as the recipient; and an antigen that affects (e.g. stops or at least hinders) the life cycle of a parasite causing the disease to be treated—such as a transmission-blocking immunogen as the antigen.

Another preferable combination is when the recipient is a human; the transgenic organism is a transgenic mosquito; and the antigen is a malarial vaccine.

Preferably the gene that is capable of expressing the desired antigen is placed under the control of a strong promoter for one of the naturally immunogenic proteins of a delivery organism, e.g. the 37 kDa protein from the saliva of mosquitoes.

One of the key advantages of the present invention is that it provides a passive and sustained immunisation programme (or method).

Another key advantage is that the present invention provides a means for efficiently targeting appropriate recipients—such as targeting humans so as to vaccinate them against malaria.

Another key advantage is that the delivery system of the present invention can be used to target reservoir populations normally inaccessible to conventional immunisation programmes.

Generally, the present invention is based on the recognition that an indispensable requirement in the life-cycle of many organisms harbouring parasites (e.g. insects), including vectors of many of the diseases for which anti-parasitic vaccines are being developed, is the necessity to take repeated meals (e.g. bloodmeals) on the recipients.

The present invention is also based on the recognition that individuals, living in areas where phytophagous or haematophagous insects are prevalent, develop immune/allergic responses to the insect saliva (i.e. phytophagous or haematophagous insects can deliver immunogenic amounts of protein to humans).

In this regard, it has been found that the saliva of a wide range of haematophagous insects contains inhibitors of T cell activation, neutrophil activity and macrophage function, as well as vasodilators and anti-inflammatory molecules (Ribeiro et al, 1991;1994; Bissonnette et al 1994). Nevertheless it has been shown conclusively that over a prolonged period of time repeated low level exposure to the saliva of haematophagous insects will lead to immunity to saliva proteins in both human or animal recipients.

Wide ranging studies on the allergic reaction to mosquito bites attest to the immunogenic potential of immunogens in insect saliva (Frazier, 1987). In Aedes it has been shown that sera from naturally exposed adults recognised up to 4 saliva proteins (Brummer-Korvenkontio et al, 1994). The dominant responses were of the IgG4, IgE and IgG1 isotypes and directed against a protein of ‘36 kDa’.

The present invention is also based on the recognition that recombinant parasite antigens expressed in insect cells can be highly and appropriately immunogenic.

The present invention is also based on the recognition that the ability to construct recombinant insects is now possible as stable transgenesis has been achieved in insects such as Drosophila.

Thus, in a highly preferred embodiment, the present invention makes use of phytophagous or haematophagous organisms (e.g. insects that usually harbour parasites) as vehicles to deliver antigens directly to both human and animal populations when bloodfeeding. The vaccination programm of the present invention could, in some instances, be used as either a substitute or an adjunct to a conventional immunisation programme.

With the highly preferred delivery system of the present invention it is now possible to immunise and/or to boost constantly humans, livestock animals and other animal reservoirs by the very bite of the phytophagous or haematophagous insects responsible for transmission of the diseases suffered, or by other haematohagous insects of appropriate feeding habit.

In addition, the present invention provides transgenic phytophagous or haematophagous insects which are capable of expressing the desired antigens in their saliva so that when they take a bloodmeal a small amount of antigen is delivered that will induce, sustain or boost an immune response in the bitten ‘recipient’.

In the following commentary the present invention is discussed in more detail, in which further preferred aspects of the present invention and the advantages thereof are described.

Choice of Organism

Ideally the organism (such as a phytophagous or haematophagous insect) chosen for the delivery system of the present invention and for any transgenesis should have one or more (preferably all) of the following characteristics: be widely distributed; be of catholic bloodfeeding habit; deliver substantial quantities of saliva at the time of biting; be easily reared in the laboratory; whose eggs may be stored for long periods; should have been the subject of significant molecular biological studies and for which there should be existing techniques for either transitory or permanent genetic transformation.

Mosquitoes are therefore clearly the organisms of preference at present.

Recognising that in malaria endemic areas a human receives as many as 200 mosquito bites per night in the rainy season, the potential for delivery of proteins in the saliva by use of the delivery system according to the present invention is therefore clearly highly advantageous. In a preferred embodiment any suitable mosquito would suffice.

Hence, presently preferred organisms to prepare the delivery system according to the present invention are mosquitos of the species Anopheline or Aedine.

It is important to note that a specific organism, such as a mosquito need not necessarily be used to prepare the delivery system of the present invention on the basis that it is a vector for the parasite from which the antigen is derived.

Instead, the organism should be an organism that can feed on the ‘recipients’ to be targeted. Thus, by way of example, mosquitos that do not harbour the parasites responsible for malaria can deliver a malarial vaccine. Also, mosquitos can be used to deliver vaccines for diseases other than malaria—such as bilharzia.

Choice of Antigen

Malarial parasite antigen (ookinetes), native protein and recombinant protein are all available to determine the immune response induced by the antigenic-bite and are now in routine use in some laboratories, together with an extensive database on the immune response of mice to these proteins (recombinant and native). Transmission blocking assays for monitoring the impact of immunisation on parasite transmission (both from membrane feeds and intact mice) are also in routine use in some laboratories.

An exemplary antigen is the transmission blocking antigen Pbs21 of the rodent malarial parasite Plasmodium berghei. Pbs21 is an ookinete antigen located on the plasma membrane. It is a member of the highly conserved gene family that also encodes Pfs25 and Pfs28, the former is a human transmission-blocking vaccine candidate about to undergo field trials (TBV25H) and the latter is the homologue of Pbs21. None of these antigens is subject to natural immunisation or boost by infection and so the possibility of repeated ‘boosting’ by transgenic mosquitoes will be of importance in the field.

Native Pbs21 is a highly immunogenic protein. In this regard, as little as 5 mg in a single inoculum has induced significant tansmission blocking activity. The entire protein has been expressed in insect cells (using baculovirus vectors) and the recombinant protein (rPbs21) is equally immunogenic to the native molecule in the absence of adjuvant (Matsuoka et al, 1994; Margos et al, 1994). Transmission is usually reduced by >90%, and so far the recombinant antigen has not been reported to have a negative impact—i.e. an enhanced transmission of the parasite. rPbs21 is therefore an ideal recombinant parasite protein for laboratory studies to show that the delivery system according to the present invention can induce an effective immune response.

Choice of Promoter

James et al (1991) have cloned the Aedes gene encoding a major saliva protein of ‘37kDa’, the probable target of the immune response seen by Brummer-Korvenkontio et al (1994). The signal sequence and putative promoter of this protein are therefore available. In a preferred embodiment, an antigen of choice is placed under the control of a strong promoter from one of the naturally immunogenic saliva proteins, e.g. the 37 kDa protein. This preferred embodiment leads to high yields of antigen that are transmitted to the recipient, for example after a bite.

Choice of Construct

Constructs of the gene are available which are capable of expressing all or selected, T and B cell epitopes of the molecule. Other preferred constructs comprise a native signal sequence but wherein they have the membrane anchor motif deleted.

These constructs result in the release of a soluble immunogen from insect cells (Matsuoka et al, 1994).

A wide range of high titre antibodies recognising both conformation dependent and/or independent epitopes are available, together with ELISA, ELISPOT, IFAT and immunogold techniques to detect and measure antigen expression.

Choice of Location for Transgenesis

A number of genes have been identified which are expressed in a salivary gland-specific manner in Aedes aegypti (James et al, 1989). A preferred embodiment therefore relates to the use of those genes, e.g. for the 36/7 kDa protein. In this regard, the upstream regions of these genes are known and are believed to contain the appropriate control sequences. Thus it is possible to combine large segments of the 5′ regions of salivary gland-specific genes with specific immunogenic domains of the Pbs21 gene. For the targeting of Pbs21 to the salivary duct of the transgenic insect in some instances it may be necessary inter alia to replace the Pbs21 signal sequence with that from a secreted salivary protein e.g. the 36 kDa protein. These combined cassettes can then be introduced into transfection vectors.

Choice of Vector

The vectors that may be used to create the delivery system according to the present invention can be vectors that are already known. For example, typical DNA vectors which may be used to introduce and express DNA in either cultured mosquito cells or mosquito embryos can be found in Lycett and Crampton (1993) or Miller et al (1987), McGrane et al (1988) and Morris et al (1989) respectively.

Choice of Cell Culture System

In the cell culture systems, cells can be transfected with DNA either transiently or stably and the introduced DNA expressed under the control of appropriate promoters (Lycett et al, 1992; Lycett and Crampton, 1993).

Choice of Transfection Method

It is now possible to modify mosquito salivary glands and mid-guts by transient transfection methods. Also, techniques have been developed for introducing DNA into mosquito embryos and, although integration does not occur at high frequency, the introduced DNA does occasionally integrate into the recipient genome so that it is passed-on to subsequent generations of the mosquito (Miller et al 1987; McGrane et al, 1988; Morris et al, 1989). Techniques for transformation of Ae. aegypti cultured cells (Lycett and Crampton (1993) Gene vol 136 pp129-136) and, more recently, organs (salivary glands, midgut, fat body) are known in the art, together with assays to characterise gene expression in these systems. Transposable element-based expression vectors may be used to achieve stable transformation of the Ae. aegypti genome. These vectors may comprise the Hermes (Jasinskiene et al., (1998) Proc. Nat. Acad. Sci. vol 95 pp3743-3747) or Mariner (Coates et al., (1998) Proc. Nat. Acad. Sci. vol 95 pp3748-3751) transposable elements. The cinnabar eye colour marker gene isolated from D. melanogaster (Cornel et al., (1997) Insect Biochem. Mol. Biol. vol 27 pp993-997) may also be useful in conjunction with these systems.

In relation to the present invention, it is possible to use both cell transfection assays and the transfection of isolated mosquito salivary glands. However, in the latter case the excised glands are relatively impermeable to transfected DNA because the basement membrane acts as a barrier. But if the glands are incubated in 0.2% elastase, this removes the basement membrane such that DNA may be transiently transfected into the salivary gland using the cationic lipid, DOTAP. Using this technique heterologous reporter genes can be expressed in the salivary glands of Aedes aegypti. This aspect is a preferred method feature.

Homologous Recombination

Experiments have indicated that homologous recombination is an appropriate approach to introducing gene constructs into the germline of mosquitoes in a gene targeted fashion (Comley et al, 1994).

It is therefore possible to introduce the exemplary Pbs21 gene under an appropriate promoter control into mosquito embryos by micro-injection to create a transgenic mosquito line expressing Pbs21 in its saliva. Simple in vitro assays, and in vivo assays based around the spit-blot system (Billingsley et al, 1991) can then be used to measure antigen delivery when a mosquito feeds.

In summation therefore the present invention diverges from the past effort that was directed towards developing a much greater understanding of the molecular biology of insect vectors (Crampton. 1994b)—such as using transgenic techniques to create mosquitoes or other vectors which are incapable of transmitting pathogens, such as the malaria parasite (Crampton et al, 1993; Muller et al, 1993; Crampton, 1994a).

In direct contrast to the previous approaches, the present invention does not render the organism refractory to the infection.

Instead, the present invention utilises organisms (such as insects) and their feeding behaviour in a novel and inventive manner to induce or boost immunity in the recipient—such as to interrupt pathogen life cycles.

In this regard, an important aspect of the delivery system of the present invention, and the transgenic organism for use therein, is the fact that the organism contains, and/or produces in situ, the vaccine outside of the recipient, and then the transgenic organism itself delivers the vaccine to the recipient.

Thus in its broadest sense the present invention relates to a delivery system for a recipient comprising an organism containing a vaccine for the recipient, wherein the vaccine is capable of being transmitted by and from the organism to the recipient, and wherein the vaccine has been prepared outside of the recipient.

A preferred embodiment of the present invention comprises a transgenic phytophagous or haematophagous insect having salivary glands comprising a gene capable of expressing a desired antigen.

A highly preferred embodiment of the present invention is a delivery system for a recipient, the delivery system comprising an organism containing a vaccine for the recipient; wherein the vaccine is capable of being transmitted by and from the organism to the recipient, wherein the vaccine is expressed by a gene contained within the organism, wherein the vaccine has been prepared outside of the recipient, and wherein the organism is an phytophagous or haematophagous organism (which may not necessarily harbour a parasite) that usually ‘feeds’ on the recipient.

The invention will now be further described by way of Examples, which assist one of ordinary skill in the art in carrying out the invention and are not intended in any way to limit the scope of the invention.

In the following examples, reference is made to the exemplary Plasmodium berghei malaria transmission-blocking antigen Pbs21 and Aedes aegypti and Anopheles stephensi. However, the following teachings are equally applicable for the creation of other delivery systems according to the present invention.

### EXAMPLE 1

Mosquito Delivery System

To demonstrate the effectiveness of the delivery system according to the present invention using mosquitoes as the delivery system organism it is desirable to follow at least some or all of the stages of the following protocol:

Protocol

1. Introduce a nucleic acid construct comprising a gene coding for the transmission-blocking antigen Pbs21 into cultured mosquito cells and express the gene in both typical transient and stable expression systems.

2. Deliver repeated and appropriately low levels of recombinant Pbs21 produced either in baculovirus or from mosquito cells to laboratory mice to assess the immunity generated. This can be conducted both on naive mice, and mice previously immunised with Pbs21 (to test for boosting of the response).

3. Transfect mosquito salivary glands in culture with nucleic acid vector constructs containing the gene coding for Pbs21 under the control of constitutive or salivary gland specific promoters.

4. Transform mosquito embryos with suitable gene constructs to create a transgenic mosquito capable of expressing in appropriate quantities the gene for the highly immunogenic protein Pbs21 in the mosquito salivary land.

5. Using mice, test the effectiveness of the recombinant Pbs21 as an immunogen when delivered by the bite of the transfected mosquitoes in laboratory studies. This could be accomplished by challenging the mice with malarial parasite and/or extracting serum and testing for immuno-response to Pbs21immunogen. The effectiveness of the recombinant Pbs21 as an immunogen may be assessed by monitoring the transmission-blocking efficacy; here the immunised mice are given a challenge infection and their ability to transmit malaria to naive mosquitoes is assayed (Tirawanchal et al, 1991).

6. Release appropriate effective transgenic mosquitos into the field to impact upon, for example, a human disease of relevance, such as malaria.

Expression and Secretion of Pbs21 by Mosquito Cells

Intact Pbs21, as well as mutants, in which the signal or anchor sequences have been deleted are expressed using baculovirus expression systems in Spodoptera cells (eg. Sf9 cells) and Heliothis larvae (Matsuoka et al, 1994; Margos et al, 1994). Using similar procedures, mosquito cells express and secrete soluble, correctly folded and modified, highly immunogenic Pbs21.

As a check, one can use the entire Pbs21 coding region and an ‘anchor-less’ construct (Pbs21c2) to test transfected Aedes aegypti and Anopheles stephensi cell lines using extant vectors under the control of the Drosophila heat shock (hsp70) and actin 5C promoters (Lycett and Crampton, 1993), transient expression systems in excised salivary gland, again using the heat shock and actin promoters from Drosophila and in transfected Aedes embryos and trausgenic insects.

In one aspect, the polypeptide(s) of interest (eg. saliva proteins, Pbs21 protein) are placed under the control of a specific promoter and signal sequences to provide appropriate expression and polarity of secretion in vivo. Preferred signal and promoter sequences are obtained from genes expressed in the salivary gland e.g. that from the 36 kDa protein (James et al 1991).

Monitoring Pbs21 Expression

Typical techniques to monitor Pbs21 expression are known.

For example, Pbs21 expression is examined both at the level of mRNA using in situ hybridisation techniques (Thompson & Sinden, 1994) and at the protein level using ELISA and IFAT/confocal scanning microscopy—thereby providing both quantitative and histological localisation of mRNA and protein expression. ELISA capture assays on whole body, or organ homogenates use two distinct monoclonal antibodies 12.1 and 13.1 (Tirawanchai et al, 1991); for IFAT assays, whole mosquitoes and/or isolated organs can be prepared are described by Simonetti et al (1993).

If the salivary gland tissues express Pbs21 ex vivo or in vivo then spit blot analysis (Billingsley et al, 1991) are used to determine whether saliva, delivered by the bite of recombinant Aedes, delivers detectable or quantifiable levels of antigen.

Immunogenicity of Expressed Pbs21 in Mice

a. Recombinant Pbs21 is delivered to mice in separate experiments by the I.V. (Anopheles) and Sub. Cut. (Aedes) routes. In different experiments the dose administered reflects weekly mosquito biting rates ranging from 5 to 1000 bites. The results show that as few as 50 bites of Aedes over a three week period induce a detectable immune response to its saliva in mice.

Recognising that all transmission-blocking effector mechanisms described to date are antibody based (Ranawaka et al, 1994a-c) the immune response is monitored in all experiments for one or more of the following properties:

i) Total anti-Pbs21 antibody titre using rPbs21 in a capture assay (Tirawanchai et al 1991);

ii) Transmission-blocking efficacy; here the immunised mice are given a challenge infection and their ability to transmit malaria to naive mosquitoes is assayed (Tirawanchai et al, 1991).

b. The experiment is repeated to determine whether administration of repeated small doses of antigen boost effectively the immune response of mice previously given a known effective immunisation with rPbs21 (see Matsuoka et al, 1994; Margos et al, 1994).

Following primary immunisation, the immune response is followed until a decline in total antibody titre is detected (usually 5-9 weeks after the last immunisation (Margos et al, 1994)). At this time immunised mice are exposed to known doses of recombinant antigen over extended periods—to simulate natural exposure to transgenic mosquitoes. The immune response of the challenged and boosted mice is followed to determine whether, and at what frequency, the immunisations are able to sustain or boost the antibody titre at levels that correlate with protection (i.e. 100 mg/ml or higher). If they are sustained, the experiments enable one to determine the duration of the sustained immune response. Following the construction of transgenic mosquitoes expressing Pbs21 in their saliva all experiments in a. and b. (supra) are repeated using transgenic mosquitoes as the source of antigen. In an alternative, it is feasible to repeat selected aspects of 2a/b using mixtures of mosquito saliva and rPbs21 from Heliothis larvae.

### EXAMPLE 2

Expression of Antigen in Transgenic Insects

Expression of Pbs21 Antigen in Transgenic Insect Tissue

Pbs21 is a transmission-blocking antigen of the rodent malaria parasite. Pbs21 is an ookinete surface protein of Plasmodium berghei. It is a highly immunogenic protein which can induce a significant transmission blockade.

A form of this Pbs21 gene lacking the C-terminal membrane GPI-anchor motif is cloned into P-element based plasmids. An appropriate promoter is selected to promote salivary gland specific expression in the transgenic flies. The Pbs21 gene is placed under the control of either the hsp70 promoter or the salivary gland specific promoter E8 from the Drosophila lysozyme gene (Schnewly (1987) Nature vol 325 pp816-818).

Drosophila melanogaster is transformed by embryo micro-injection of said plasmid(s) (Spradling, A. C. and Rubin, G. M. (1982). Science vol. 218 pp341-347; Rubin. G. M. and Spradling, A. C. (1982). Science vol. 218 pp348-353).

Several stable transgenic lines of D. melanogaster are established by means of P element transposition. All such transgenic flies are found to be expressing the parasite Pbs21 protein in an immunoreactive and potentially immunogenic conformation.

The transposition of the transposon(s) into the fly genome(s) does not appear to adversely affect the flies' fitness, fecundity or fertility. The transposons are maintained over several generations and transgene protein is expressed in such flies up to at least the 32nd generation.

High levels of Pbs21-A are expressed in Drosophila melanogaster stable transformants, and the protein is recognised by conformation dependent monoclonal antibodies.

The immunogencity of Pbs21-A expressed in flies is tested by immunising mice with homogenates of said transformed flies.

The solubility and secretion of the antigen in the insects' saliva is examined.

In this Example, three sets of transformed flies are produced according to the invention;

1. tra.hs.Pbs21: These transformed flies express the fill length Pbs21 protein from the hsp70 promoter. Protein expression is induced by heat shock. Protein is detected by western blot analysis and quantified in individual flies. Homogenates of these transformants are used to immunise mice.

Two out of the four mice immunised with tra.hs.Pbs21 develop an anti-Pbs21 immune response two weeks after the third immunisation. This result conforms the strong immunogenicity of this antigen.

It is estimated that each transformed fly contains approximately 70 ng of protein.

2. tra.hs.Pbs21-A, The Pbs21-A DNA construct sequence of the P-element transposition/transformation plasmid used in this Example is tested for expression in a Baculovirus insect cell expression system. The transformed flies express the soluble (anchorless) Pbs21-A protein, having the C-terminal GPI anchor domain truncation, from the hsp70promoter.

In the same way as for tra.hs.Pbs21 flies, high producing lines are selected by western blotting comparison.

The protein is detected in western blots and its level of expression is in the same range as for tra.hs.Pbs21, approximately 70 ng per transformed fly.

3. tra.E8.Pbs21-A; The E8 sequence is a sequence upstream of the Drosophila lysozyme P gene, which drives salivary gland specific expression in D. melanogaster. This E8 promoter sequence sequence is incorporated into a transgene upstream of the Pbs21-A coding sequence. A polyadenylation site (from the An. gambiae trypsin gene, clone TY1C5.2) is placed downstream (3′) of Pbs21-A.

The gene cassette containing the above three sequences is cloned into a transformation vector (pCaSpeR-4) and flies are transformed.

Numerous transformant flies are obtained. IFATs on cryosections of tra.Ei.Pbs21-A flies reveal expression of Pbs21 in the salivary glands as seen in tissue sections of tra.E8.Pbs21-A flies.

Pbs21-A protein can be detected in spit blot tests, showing that saliva discharges contain the Pbs21-A protein.

These experiments show that parasite antigen can be expressed in a transgenic insect in vivo, and that antigen so expressed retains a high degree of immunogenicity.

### EXAMPLE 3

Transmission Blocking Using Antigen Expressed in Transgenic Insect Tissue

Pbs21 belongs to a group of malarial sexual-stage antigens with transmission-blocking potential. This group comprises two families of 21-30 kDa proteins expressed on the surface of the gamete, zygote and ookinete stages of various Plasmodium species (for example see Kaslow et al., (1988) Nature vol 333 pp74-76; Kaslow et al., (1989) Mol. Biochem. Parasitol. vol 33 pp283-288; Paton et al., (1993) Mol Biochem Parasitol vol 59 pp263-276; Duffy et al., (1993) J. Exp. Med. vol 177 pp505-510).

A gene encoding Pbs21 has been cloned and sequenced (Paton et al., (1993) Mol Biochem Parasitol vol 59 pp263-276).

A Pbs21 gene is introduced into both Drosophila and Aedes systems, followed by immunisation of mice with this recombinant product to produce a transmission-blocking immunity.

Expression of Pbs21 in D. Melanogaster

The gene encoding Pbs21 is cloned into the P element based plasmid CaSpeR-hs. This comprises the Drosophila Heat Shock Protein 70 (hsp70) promoter together with a white (w) eye colour selectable marker gene, flanked by inverted terminal repeats (Schnewly (1987) Nature vol 325 pp816-818).

This plasmid is introduced into the D. melanogaster genome by embryo microinjection in order to achieve stable germ line integration. Two forms of the Pbs21 gene are expressed, one encoding the full length membrane-bound protein (Pbs21) and the other, Pbs21-A, encoding a truncated secreted/soluble protein deleted at the 3′ end.

Nineteen first generation Pbs21 transformants and thirty-one first generation Pbs21-A transformants are produced. Recombinant Pbs21 and Pbs21-A is detected by western blot analysis in all permanently established homozygous fly lines (16 for Pbs21, 25 for Pbs21-A).

Lines are selected that produce high levels of antigen, typically approx. 72-73 ng of protein per fly. These lines are used to determine the copy number of the inserted transgene using Southern hybridisation analysis. The location of these inserts is also examined using in situ hybridisation on polytene chromosomes. No evidence is found that expression of the parasite Pbs21 protein is detrimental to the flies.

To investigate the stability of the insertion over time, genomic DNA is isolated from generations 4, 8 and 12 of each of the transgenic lines of Pbs21 or Pbs21-A transformed flies. Southern hybridisation analysis of this DNA with a digoxigenin-labelled Pbs21 probe produces a consistent signal throughout the generations, confirming that the integration of the transposable element transgene into the insects' genome is stable over at least this number of generations.

Western blots on fly homogenates demonstrate that protein expression is similarly consistent. Homogenates of these flies induce an immune response to Pbs21.

These data show that the Pbs21 gene can be stably introduced into the D. melanogaster genome.

Expression of Pbs21 in Salivary Glands.

It is desirable to target transgene antigen expression to the salivary glands.

A 700 bp fragment from the promoter region of the Lysozyme P (Lys P) gene from D. melanogaster (E8) is fused to the bacterial LacZ gene in the vector pCaSpeR-AUG-B-gal and this construct used to transform D. melanogaster embryos.

Six different transformants are obtained, all exhibiting salivary gland-specific expression of the LacZ gene, thereby demonstrating that this region can act as a functional salivary gland-specific promoter in Drosophila. The Pbs21-A encoding sequence is now placed downstream of this promoter in the plasmid pCaSpeR4 and this is used to transform D. melanogaster embryos.

Expression of Pbs21 in Aedes aegypi

The full length Pbs21 encoding sequence is placed under the control of the Bombyx mori baculovirus immediate-early gene promoter in the expression vector pVJ12-IEGprom (Vanden Broeck (1995) J. Neurochem. vol 64 pp2387-2395). This construct (pVJ12-pbs21) is expressed in cultured Ae. aegypti cells (line Mos20).

Mos20 Transfections:

Exponentially growing Ae. aegypti cells are seeded into 50 cm³ flasks containing complete MM/VP12 medium and maintained at 28° C. for 24 hours until 60-70% confluent. The medium is then replaced with 2.0 ml of serum-free MMIVP12 containing polybrene (12.5 mg/ml) and 15 mg of either pVJ12-IEEGprom or pVJ12-IEG prom only, and incubated for 6 hours. After this period, glycerol is added to a final concentration of 10% (v/v) and incubated for 3 minutes. Cells are then washed once with PBS and incubated in complete medium to recover.

Pbs21 mRNA is detectable by northern blotting 12 hours post-transfection and recombinant Pbs21 is detectable after 24 hours. Recombinant Pbs21 in the form of crude cell lysates plus adjuvant is used to immunise BALB/c mice subcutaneously three times, at 14 day intervals.

Immunisation Protocol:

Groups of 3 BALB/c mice are immunised 4 times at 2 week intervals with Ae. aegypti cells expressing recombinant Pbs21. The first 2 immunisations are made subcutaneously, followed by 1 intravenous immunisation into the tail vein and a final subcutaneous immunisation. Adjuvant is added to samples administered subcutaneously. Control mice are similarly immunised with cells tanfected with pVJ12-IEGprom without the pbs21 gene. Sera are collected from the tail vein 10 days after each immunisation and tested by Western Blot analysis for the presence of anti-Pbs21 antibodies. Mice are bled 14 days after the final immunisation and sera stored at −70° C.

Native crude ookinete proteins (containing pbs21 ) are fractionated by PAGE on a 10% gel. The proteins are transferred to a nitrocellulose filter by Western blotting and used to detect the presence of anti-pbs21 antibodies. The presence anti-pb21 antibodies is detected using alkaline phosphatase labelled goat anti-mouse IgG and developed with NBT and BCIP.

Antibody responses are detectable by western blotting after the second immunisation.

Control mice are similarly immunised with crude lysates from cells not expressing Pbs21. After the third immunisation, mice are bled and sera pooled and used in a transmission-blocking assay.

Transmission-Blocking Assay:

P. berghei ANKA clone 2.34 is maintained in outbred Theiler's Original mice and transmitted through Anopheles stephensi mosquitoes. Theiler's Original mice are infected with 10⁷ parasites, intraperitoneally. Parasitaemias indicating the course of infection are monitored on Giemsa-stained thin films made from tail blood. For the membrane feed, 0.4 ml of heparinised infected blood from mice bled on day 4 of infection is mixed with 0.2 ml of test or control serum in a membrane feeder at 19° C. An uninfected feed is administered 3 days later. Mosquito midguts are dissected and oocyst counts are performed on day 10.

The percentage transmission (% Tm) and percentage blockade (% Bl) are calculated from the following equations:

$$\begin{matrix}\% Tm = \frac{Arithmetic mean no . of oocysts per gut in experimental group}{Arithmetic mean no . of oocysts per gut in control group} \times 100 \\ \% BI = ( 100 - ( \% Tm ) )\end{matrix}$$

Using these methods, it is demonstrated that a transmission-blockade of 71.8% is induced together with a 28% reduction in prevalence of infection when compared with control values (Table 1).

TABLE 1
Results of transmission blocking assay using
antigen produced in mosquito cells

| | Cells not expressing | Cells expressing |
|---|---|---|
| | Pbs21 | Pbs21 |
| Number of infected | 44/50 | 30/50 |
| mosquitoes/number | | |
| dissected | | |
| Prevalence of infection (%) | 88% | 60% |
| Geometric mean number of | 21.0 ± 3.2 | 7.66 ± 2.33 |
| oocysts per mosquito (±SE) | | |
| Arithmetic mean number of | 23.9 ± 4.5 | 6.7 ± 1.94 |
| oocysts per mosquito (±SE) | | |

Expression in Mosquito Salivary Glands

Mosquito salivary glands may be transfected and maintained in culture (Morris et al., (1995) Am. J. Trop. Med. Hyg. vol 52 pp456-460).

Following transfection of mosquito salivary glands, the Pbs21 gene is expressed transiently in cultured glands. Pbs21 mRNA is first detectable by in situ techniques 12 hours post-transfection, with the highest levels of expression in periphery.

Recombinant protein is first detectable by immunohistochemistry 24 hours after transfection with the pattern of expression correlating well with that of the mRNA. In contrast to Pbs21, Pbs21-A is shown to be secreted into the cell supernatant. These cell supernatants are concentrated, BALB/c mice are given 3 immunisations, one intravenously followed by two intramuscularly, without adjuvant. Antibodies recognising native ookinete-derived antigen are first detectable by western blot analysis after the third immunisation.

The present invention therefore relates to a delivery system for a recipient, the delivery system comprising an organism containing a vaccine for the recipient, wherein the vaccine is capable of being transmitted by and from the organism to the recipient, and wherein the vaccine is prepared outside of the recipient. Preferably, the organism is one that is antagonistic towards a subject, such as towards a human.

Thus, the present invention provides a vaccine delivery system comprising an organism containing and/or being capable of producing a vaccine for a recipient, wherein the vaccine is capable of being transmitted by and from the organism to the recipient, and wherein the vaccine is prepared outside of the recipient. The present invention also provides a vaccine delivery system for a recipient, the delivery system comprising an organism containing and being capable of producing a vaccine for the recipient, wherein the vaccine is capable of being transmitted by and from the organism to the recipient and wherein the vaccine is produced by expression of a suitable gene therefor outside of the recipient.

In summation, the present invention will now be described by way of numbered paragraphs.

1. A delivery system for a recipient, the delivery system comprising an organism containing a vaccine for the recipient, wherein the vaccine is capable of being transmitted by and from the organism to the recipient, and wherein the vaccine is prepared outside of the recipient.

2. A delivery system according to paragraph 1 wherein the organism is a transgenic organism and the vaccine is expressed by a gene contained within the transgenic organism.

3. A delivery system according to paragraph 2 wherein the gene is stably incorporated in the genome of the transgenic organism.

4. A delivery system according to any one of the preceding paragraphs wherein the recipient is a livestock animal, a human or a plant.

5. A delivery system according to any one of the preceding paragraphs wherein the organism has a mouth and the vaccine is transmitted via the mouth of the organism.

6. A delivery system according to any one of the preceding paragraphs wherein the organism is an organism that usually harbours a parasite.

7. A delivery system according to any one of the preceding paragraphs wherein the organism is an phytophagous or haematophagous organism.

8. A delivery system according any one of the preceding paragraphs wherein the organism is an insect, preferably an haematophagous insect.

9. A delivery system according to paragraph 8 wherein the haematophagous insect is a mosquito.

10. A delivery system according to any one of the preceding paragraphs wherein the organism is an organism that in its native state would normally feed on the recipient and in doing so transfer an infectious agent to the recipient and thereby cause an infection.

11. A transgenic organism comprising a gene capable of expressing a vaccine wherein the transgenic organism itself is suitable for use as a means of delivering the vaccine to a recipient for the vaccination thereof, and wherein the vaccine is prepared outside of the recipient.

12. A transgenic organism according to paragraph 11 wherein the gene is stably incorporated in the genome of the transgenic organism.

13. A transgenic organism according to paragraph 11 or paragraph 12 wherein the transgenic organism is that defined in any one of paragraphs 5-10.

14. A transgenic organism according to any one of paragraphs 11 to 13 wherein the recipient is a livestock animal, a human or a plant.

15. A vaccine derived from a transgenic organism according to paragraph 11 or any paragraph dependent thereon.

16. Use of a transgenic organism according to paragraph 11 or any paragraph dependent thereon for the manufacture of a vaccine for the vaccination of a recipient by the transgenic organism, wherein the vaccine is delivered to the recipient by the transgenic organism, and wherein the vaccine is prepared outside of the recipient.

17. A use according to paragraph 16 wherein the recipient is a livestock animal, a human or a plant.

18. A transgenic phytophagous or haematophagous insect having salivary glands comprising a gene capable of expressing a desired antigen to produce immunity.

19. A transgenic phytophagous or haematophagous insect according to paragraph 18 wherein the insect is a mosquito.

20. A tansgenic phytophagous or haematophagous insect according to paragraph 18 or paragraph 19 wherein the antigen is a malarial vaccine.

21. A transgenic phytophagous or haematophagous insect according to any one of paragraphs 18 to 20 wherein the gene codes for an antigen that affects the life cycle of a parasite causing the disease to be treated- such as a transmission-blocking immunogen.

22. A recipient comprising the saliva of a transgenic organism according to paragraph 18 or any paragraph dependent thereon wherein the saliva contains an antigen produced by expression of the gene coding for the same.

23. A recipient according to paragraph 22 wherein the recipient is a human. the transgenic organism is a transgenic mosquito and the antigen is a malarial vaccine. such as a transmission-blocking immunogen.

24. A method of vaccinating a recipient comprising exposing the recipient to a transgenic organism according to paragraph 11 or any paragraph dependent thereon and allowing the vaccine to be transmitted by and from the transgenic organism to the recipient, wherein the vaccine is prepared outside of the recipient.

25. A method according to paragraph 24 wherein the recipient is a livestock animal, a human or a plant.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry, biotechnology and/or molecular biology or related fields are intended to be within the scope of the following claims.

### REFERENCES

Beard, C. B., et al (1993) Genetic transformation and phylogeny of bacterial symbionts from tsetse. Insect Molecular Biology 1, 123-131.

Billingsley, P. F., et al (1991). Detection of mature malaria infections in live mosquitos. Trans. R. Soc. Trop. Med. Hyg. 85, 450-453.

Bissonnette, E. Y., et al (1993). Extracts of mosquito salivary gland inhibit tumour necrosis factor alpha release from mast cells. Parasite Immunology 15, 27-33.

Brummer-Korvenkontio, H., et al (1994) Detection of mosquito-saliva specific IgE and IgG4 antibodies by immunoblotting. J. Allergy and Clin. Immunol. 93, 551-555.

Collins, F. H. (1994). Prospects for malaria control through the genetic manipulation of its vectors. Parasitology Today 10, 37-371.

Crampton, J. M. (1994a). Approaches to vector control: New and Trusted. 3. Prospects for the genetic manipulation of insect vectors of disease. Trans. R. Soc. Trop. Med. Hyg. 88, 141-143.

Crampton, J. M. (1994b). Molecular studies of insect vectors of malaria. Adv. Parasitol. 34, 1-31.

Crampton, J. M. et al (1990a). Transgenic mosquitos: a future vector control strategy. Parasitol. Today 6, 31-36.

Curtis, C. F. (1994). The case for malaria control by genetic manipulation of its vectors. Parasitology Today 10, 371-374.

Gwadr, R. W. (1994). Genetic approaches to malaria control: How long the road? Am. J. Trop. Med. Hyg. 50, 116-125.

James, A. A., et al (1989). A salivary gland-specific, maltase-like gene of the vector mosquito, Aedes aegypti. Gene 75, 73-83.

Kaslow, D. C. (1993). Transmission-blocking immunity against malaria and other vector-borne diseases. Current Opinion in Immunology 5, 557-565.

Kaslow, D. C. (1992). Towards a transmission blocking vaccine for Plasmodiurn falciparum. Proceedings of the International Meeting for Tropical Medicine and Malaria, Patayya. TuS13 pp177-178.

Lycett, G., et al (1992). The expression of reporter genes in cultured cells of Aedes aegypti. In “Insect Molecular Science” (J. M. Crampton and P. Eggleston, eds.) pp. 247-48. Academic Press, London.

Lycett, G. J. et al (1993). Stable transformation of mosquito cell lines using a hsp70:neo fusion gene. Gene 1365, 129-136.

McGrane, V. et al (1988). Micro injection of DNA into Aedes triseriatus ova and detection of integration. Am. J. Trop. Med. Hyg. 39, 502-510.

Margos, G. et al. (1994). Expression of the ookinete antigen Pbs2l of Plasmodium berghei in a baculovirus-insect cell system produces an efficient transmission blocking immunogen. In press.

Mastsuoka, H. et al. (1994). Induction of anti-malarial transmission blocking immunity with a recombinant ookinete surface antigen of Plasmodium berghei produced in silkworm larvae using the baculovirus expression vector system. In press.

Miller, L. H. et al (1987). Stable integration and expression of a bacterial gene in the mosquito Anopheles gambiae. Science 237, 779-781.

Morris, A. C. et al. (1989). Genetic transformation of the mosquito Aedes aegypti by micro-injection of DNA. Medical and Veterinary Entomology 3, 1-7.

Muller, H. M. et al (1993). Members of a trypsin gene family in Anopheles gambiae are induced in the gut by blood meal. EMBO Journal 12, 2891-2900.

Ranawaka, G., et al (1993). The effect of transmission-blocking antibody ingested in primary and secondary blood feeds, upon the development of Plasmodium berghei in the mosquito vector. Parasitology, 107, 225-231.

Ranawaka, G., et al (1994). Characterisation of the effector mechanisms of a transmission-blocking antibody upon differentiation of Plasmodium berghei gametocytes into ookinetes in vitro. Parasitology, 109, 11-17.

Ranawaka, G., et al. (1994). Characterisation of the modes of action of anti Pbs21 malaria transmission-blocking immunity upon ookinete of oocyst differentiation in vivo. Parasitology. (In Press).

Robertson, H. M. et al (1993). The mariner transposable element is widespread in nature. Nature 362, 241-245.

Ribeiro, J. M. C. et al (1990). Saliva of the tick Ixodes dammini inhibits neutrophil function. Experimental Parasitology, 70, 382-389.

Ribeiro, J. M. C. et al (1994). Salivary vasodilators of Aedes triseriatus and Anopheles gambiae (Diptera: Culicidae). Journal of Medical Entomology 31, 747-753.

Sinden, R. E. (1994). Antimalarial transmission-blocking vaccines. Science Progress, 77.

Spielman, A. (1994). Why entomological antimalaria research should not focus on transgenic mosquitos. Parasitology Today 10, 374-376.

Thompson and Sinden (1994). In situ detection of RNA during sexual development of Plasmodiun berghei. Molecular and Biochemical Parasitology (In press).

Tirawanchai, N. et al (1991). Analysis of immunity induced by the affinity purified 21 kilodalton zygote-ookinete surface antigen of Plasmodium berghei. Infect. Immunity 59, 3644.

Warren, A. M. et al (1991). The Aedes aegypti genome: complexity and organisation. Genetical Research (Cambridge) 58, 225-232.

Warren, A. M. et al (1994). Mariner: Its prospects as a transformation vector for medically important insects. Parasitol. Today 11, 58-63.

1. A delivery system for a recipient, the delivery system comprising a transgenic organism containing a vaccine for the recipient, wherein the vaccine is expressed by a gene contained within the transgenic organism, and the vaccine is capable of being transmitted by and from the organism to the recipient, and wherein the vaccine is prepared outside of the recipient, and wherein the recipient is an animal.

2. A delivery system according to claim 1 wherein the animal is a livestock animal or a human.

3. A delivery system for a recipient, the delivery system comprising a transgenic phytophagous or haematophagous insect such as a mosquito containing a vaccine for the recipient, wherein the vaccine is expressed by a gene contained within the transgenic organism, and the vaccine is capable of being transmitted by and from the organism to the recipient, and wherein the vaccine is prepared outside of the recipient, and wherein the recipient is an animal.

4. A delivery system according to claim 3 wherein the animal is a livestock animal or a human.

5. A delivery system according to claim 1 wherein the organism is an organism that in its native state would normally feed on the recipient and in doing so transfer an infectious agent to the recipient and thereby cause an infection.

6. A transgenic organism comprising a gene capable of expressing a vaccine wherein the transgenic organism itself is suitable for use as a means of delivering the vaccine to a recipient for the vaccination thereof, and wherein the vaccine is prepared outside of the recipient.

7. A transgenic phytophagous or haematophagous insect having salivary glands comprising a gene capable of expressing a desired antigen to produce immunity.

8. A transgenic phytophagous or haematophagous insect having salivary glands comprising a gene capable of expressing a desired antigen to produce immunity, wherein the insect is a mosquito.

9. A transgenic phytophagous or haematophagous insect having salivary glands comprising a gene capable of expressing a desired antigen to produce immunity, wherein the insect is a mosquito and wherein the antigen is a malarial vaccine.

10. A transgenic phytophagous or haematophagous insect having salivary glands comprising a gene capable of expressing a desired antigen to produce immunity, wherein the insect is a mosquito, and wherein the gene codes for an antigen that affects the life cycle of a parasite causing the disease to be treated—such as a transmission-blocking immunogen.

11. A transgenic phytophagous or haematophagous insect having salivary glands comprising a gene capable of expressing a desired antigen to produce immunity, wherein the insect is a mosquito, wherein the gene codes for an antigen that affects the life cycle of a parasite causing the disease to be treated, such as a transmission-blocking immunogen, and wherein the saliva contains an antigen produced by expression of the gene coding for the same.

12. A method of vaccinating a recipient wherein the vaccine is prepared outside of the recipient, comprising exposing the recipient to a transgenic organism comprising a gene capable of expressing a vaccine and allowing the vaccine to be transmitted by and from the transgenic organism to the recipient.

13. A method of vaccinating a recipient wherein the vaccine is prepared outside of the recipient, comprising exposing the recipient to a transgenic organism comprising a gene capable of expressing a vaccine and allowing the vaccine to be transmitted by and from the transgemc organism to the recipient, wherein the recipient is an animal.

14. A method according to claim 13 wherein the animal is a livestock animal or a human.
